(19) United States
(12) Patent Application Publication
(10) Pub. No.: US 2025/0163050 A1
(43) Pub. Date: May 22, 2025

(54) CRYSTALLINE (+)-TETRABENAZINE

(71) Applicant: Foresee Pharmaceuticals Co., Ltd., Taipei City (TW)

(72) Inventors: Yuhua LI, Newark, DE (US); Shih-fan JANG, Taipei City (TW); Wen-yen HUANG, Taipei City (TW); Ming-hsin LI, Taipei City (TW)

(21) Appl. No.: 18/836,650

(22) PCT Filed: Feb. 15, 2023
(86) PCT No.: PCT/US2023/062621

§ 371 Date: Aug. 7, 2024

Related U.S. Application Data
(60) Provisional application No. 63/268,017, filed on Feb. 15, 2022.

(51) Int. Cl. C07D 471/04; A61K 31/4745; A61K 47/10; A61K 47/14; A61K 47/22; A61K 47/26; A61K 47/34

(52) U.S. Cl. CPC C07D 471/04; A61K 31/4745; A61K 47/10; A61K 47/14; A61K 47/22; A61K 47/26; A61K 47/34

(57) ABSTRACT

The present application relates to crystal forms of (+)-tetrabenazine. The present application also relates to a pharmaceutical composition comprising the crystal form of (+)-tetrabenazine, as well as methods of using the crystal forms of (+)-tetrabenazine in the treatment of hyperkinetic movement disorders, and methods for obtaining such crystal forms.

### CROSS REFERENCE TO RELATED APPLICATION

This application claims priority to U.S. Provisional Patent Application No. 63/268,017 filed on Feb. 15, 2022, the disclosure of which is incorporated herein by reference in its entirety.

### TECHNICAL FIELD

The present application relates to crystal forms of (+)-tetrabenazine. The present application also relates to a pharmaceutical composition comprising a crystal form of (+)-tetrabenazine, as well as methods of using the crystal forms of (+)-tetrabenazine in the treatment of hyperkinetic movement disorders, and methods for obtaining such crystal forms.

### BACKGROUND

Tetrabenazine (TBZ), also known as Ro 1-9569, is a benzoquinolizine derivative with the chemical name 1,3,4,6,7,11b-hexahydro-9,10-dimethoxy-3-(2-methylpropyl)-2H-benzo[a]quinolizin-2-one (GB 789,789 and U.S. Pat. No. 2,830,993). TBZ exhibits a dopamine depleting effect by reversibly binding to the vesicular monoamine transporter 2 (VMAT2) to inhibit monoamine uptake into granular vesicles of pre-synaptic neurons and, thereby, augment their degradation by monoamine oxidases within the cytoplasm (Scherman, D., Jaudon, P., Henry, J., Characterization of the monoamine carrier of chromaffin granule membrane by binding of [2-3H] dihydrotetrabenazine, Proc. Natl. Acad. Sci. U.S.A., 1983, 80:584-588; Thiriot, D. S., Ruoho, E. A., Mutagenesis and Derivatization of Human Vesicle Monoamine Transporter 2 (VMAT2) Cysteines Identifies Transporter Domains Involved in Tetrabenazine Binding and Substrate Transport, J. Biol. Chem., 2001, 276:27304-27315). TBZ was approved in Britain in 1971 and more recently (Aug. 15, 2008) by the FDA in the United States as the first drug to treat chorea associated with Huntington's disease (Mestre, T. Ferreira, J., Coelho, M. M., Rosa, M., Sampaio, C., Therapeutic interventions for symptomatic treatment in Huntington's disease, Cochrane Database Syst. Rev., 2009, 3, CD006456). It is sold under the brand names Nitoman™ and Xenazine™ among others.

Although Xenazine was approved by the FDA to treat chorea associated with Huntington's disease, the safety and efficacy of the treatment is affected by dose limiting side-effects.

Specifically, the dose must be titrated for each individual patient via a prolonged dose escalation process. Escalation to the most effective dose is limited by the onset of side-effects [e.g., drowsiness (36.5%), parkinsonism (28.5%), depression (15.0%), insomnia (11.0%), nervousness or anxiety (10.3%), and akathisia (9.5%) (J. Jankovic and J. Beach, Long-term effects of tetrabenazine in hyperkinetic movement disorders, Neurology, 1997 February, 48 (2): 358-62). This process introduces a safety concern in the eliciting of side effects in each patient and limits the efficacy of the therapy in that the optimum dose may not be achieved prior to the onset of side effects. Evidence shows that the side effects associated with tetrabenazine are related to elevated peak plasma concentrations of the active metabolite.

TBZ has two chiral centers at the 3 and 11b carbon atoms and theoretically can have four isomeric forms. However, the marketed drug, Nitoman™ or Xenazine™ is only a racemate of (+)-(3R,11bR)-TBZ and (−)-(3S,11bS)-TBZ (the trans-isomers of TBZ) due to the thermodynamic instability of the cis-isomer of TBZ as shown in Scheme 1 (Johannes M., Altmann K. H., A ring-closing metathesis-based approach to the synthesis of (+)-tetrabenazine, Org. Lett., 2012 Jul. 20, 14 (14): 3752-5, 2012 Jun. 28; Yao Z, Wei X, Wu X, et al., Preparation and evaluation of tetrabenazine enantiomers and all eight stereoisomers of dihydrotetrabenazine as VMAT2 inhibitors, Eur. J. Med. Chem., 2011, 46 (5): 1841-1848).

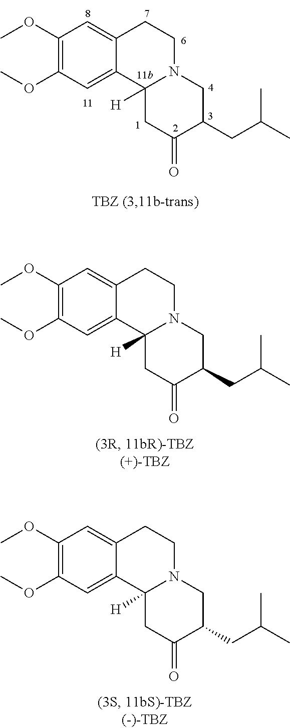

### Scheme 1. Structures of Trans-Tetrabenazine and its Enantiomers, (+)-TBZ and (−)-TBZ.

TBZ has a low and variable bioavailability. In vivo, TBZ undergoes rapid and extensive hepatic metabolism to its corresponding 2-hydroxy derivatives (dihydrotetrabenazines (DHTBZs), Scheme 2) as the pharmacologically active species. Importantly, the binding of DHTBZs to VMAT2 is highly stereospecific, with Ki values of 3.96 nM and 13.4 nM for the (+)-α- and (+)-β-dihydro derivatives, (2R,3R,11bR)-DHTBZ and (2S,3R,11bR)-DHTBZ, respectively, which are derived from (+)-TBZ. In contrast, the binding affinities for the corresponding reduction products derived from (−)-TBZ are only in the micromolar level (23.7 μM and 2.5 μM for (−)-α- and (−)-β-DHTBZ, respectively) (Yao, Z., Wei, X., Wu, X., Katz, J. L., Kopajtic, T., Greig, N. H., Sun, H., Eur. J. Med. Chem., 2011, 46; 1841; Kilbourn, M. R., Lee, L. C., Heeg, M. J., Jewett, D. M., Chirality, 1997, 9; 59. Kilbourn, M., Lee, L., Vander Borght, T., Jewett, D., Frey, K., Eur. J. Pharmacol., 1995, 278:249].

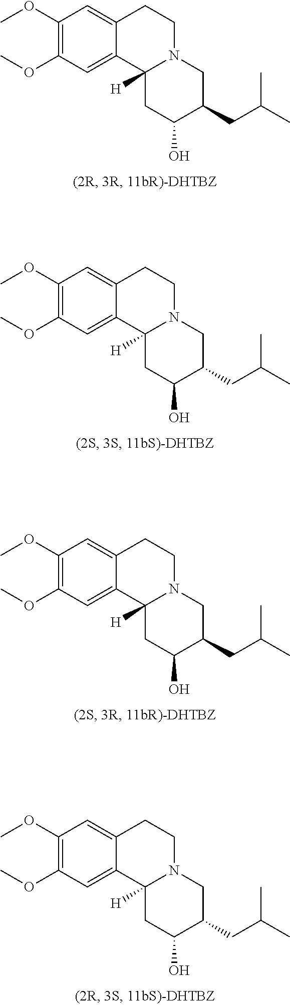

Scheme 2. Structures of the TBZ Metabolites (DHTBZ) with Trans-Configuration at C-3 vs. C-11b.

In addition, (2R,3R,11bR)-DHTBZ ((+)-α-DHTBZ) and (2S,3R,11bR)-DHTBZ ((+)-β-DHTBZ) isomers exhibit negligible binding at dopamine receptors, indicating that they might be unlikely to give rise to dopaminergic side effects encountered with (Clarke I., Turtle R., Johnston G., International Patent Application Publication No. WO2005077946). Moreover, they also lack the unwanted sedative effects associated with TBZ. The stereospecific binding profile of DHTBZ indicates that TBZ enantiomers and DHTBZ stereoisomers may have different pharmacological and/or toxicological profiles, which still remain to be determined. Therefore, it is highly desirable to develop a practical access to optically pure TBZ enantiomers and DHTBZ stereoisomers to support the development of more potent and safer drugs for the treatment of hyperkinetic movement disorders, such as chorea associated with Huntington's disease.

TBZ racemate or deuterated TBZ racemate can be in either crystalline or amorphous form. Crystalline forms of racemic TBZ are reported in WO 2012081031 A1 and WO 2015175505 A1, and those of deutetrabenazine are reported in WO 2014047167 A1 and U.S. Pat. No. 9,550,780B2). Jayachandra and co-workers disclosed methods of preparing deutetrabenazine in amorphous form, including processes for making deutetrabenazine in amorphous form (WO 2019130252 A2) via techniques such as spray drying or distillation. Tetrabenazine in amorphous form is also available commercially from Biorbyt Ltd (Cambridge, Cambridgeshire, CB4 0WY, United Kingdom).

Different crystalline forms of a drug compound may have different properties such as crystal packing, thermodynamic, spectroscopic, kinetic, surface and mechanical properties. A particular crystalline form of a drug compound may be less sensitive to heat, relative humidity (RH) and/or light, resulting in a desirable stability and shelf-life. A particular crystalline form of a drug compound may also provide more favorable flowability, compressibility and/or density properties, thereby providing more desirable characteristics for formulation and/or drug product manufacturing. A particular crystalline form of a drug compound may also have optimal solubilities, which allow for a desirable dissolution profile and/or a specific pharmacokinetic profile to be achieved.

It is well established that the prediction of whether any given compound will exhibit crystalline polymorphism or amorphism is not possible. Therefore, it is not possible to know the number and type of crystalline forms that can exist for (+)-TBZ, or the methods that will be suitable for the preparation of any given crystalline form. In addition, prediction of the properties of any unknown crystalline forms, and how they will differ from other crystalline forms of the same compound, remains elusive (Joel Bernstein, Polymorphism in Molecular Crystals, Oxford University Press, New York, 2002). Therefore, there is still a need for a novel crystalline form of (+)-TBZ for use in the preparation of drug products with improved properties.

### BRIEF SUMMARY

The objective of the present application is to provide new approaches of improving the properties of (+)-TBZ for the treatment of hyperkinetic movement disorders, as well as providing crystal forms of (+)-TBZ.

The present application provides crystal forms of (+)-TBZ, processes for preparing the crystal forms of (+)-TBZ, pharmaceutical compositions comprising the crystal form (+)-TBZ, and the use of the crystal forms of (+)-TBZ for the treatment of hyperkinetic movement disorders.

In some embodiments, the present application provides a crystal form of (+)-tetrabenazine, characterized in that the crystal form has an X-ray diffraction spectrum comprising peaks at diffraction 2θ angles of 8.6±0.2°, 14.1±0.2°, 15.0±0.2°, 17.3±0.2°, 22.6±0.2°, and 23.1±0.2°.

In some embodiments, the crystal form of (+)-tetrabenazine is Form 1.

In certain embodiments, the X-ray diffraction spectrum of Form 1 comprises peaks at 20 angles of 6.5±0.2°, 8.6±0.2°, 12.1±0.2°, 14.1±0.2°, 15.0±0.2°, 16.5±0.2°, 17.3±0.2°, 17.9±0.2°, 22.6±0.2°, and 23.1±0.2°.

In certain embodiments, the X-ray diffraction spectrum of Form 1 comprises the peaks as shown in Table 2 of the present application.

In certain embodiments, Form 1 has an X-ray powder diffraction spectrum represented by diffraction angle 2θ angle substantially as shown in FIG. 1A or FIG. 1B.

In some embodiments, the crystal form of (+)-tetrabenazine is Form 2.

In certain embodiments, the X-ray diffraction spectrum of Form 2 comprises peaks at 20 angles of 8.6±0.2°, 12.1±0.2°, 14.1±0.2°, 15.00.2°, 17.3±0.2°, 17.9±0.2°, 22.6±0.2°, and 23.1±0.2°.

In certain embodiments, the X-ray diffraction spectrum of Form 1 comprises the peaks as shown in Table 3 of the present application.

In certain embodiments, Form 2 has an X-ray powder diffraction spectrum represented by diffraction angle 2θ angle substantially as shown in FIG. 2A or FIG. 2B.

In certain embodiments, Form 2 has a differential scanning calorimetry scan spectrum comprising an endothermic peak at 115±5° C.

In certain embodiments, Form 2 has a differential scanning calorimetry scan spectrum substantially as shown in FIG. 12A or FIG. 12B or FIG. 12C.

In certain embodiments, Form 2 has a thermogravimetric analysis profile substantially as shown in FIG. 13A or FIG. 13B or FIG. 13C.

The present disclosure provides a method for preparing the crystal form of (+)-tetrabenazine, including Form 1 and Form 2.

In some embodiments, the crystal form is Form 1, and the method comprises:
a) obtaining a salt of (+)-TBZ from racemate TBZ using a resolving agent, preferably the resolving agent is (1S)-(+)-10-camphorsulfonic acid ((+)-CSA);
b) dissolving the salt of (+)-TBZ in a solvent to obtain a solution;
c) adjusting the pH of the solution with a pH adjusting agent to basic condition, preferably to pH 7.5-8.5; and
d) obtaining Form 1 by filtration.

In certain embodiments, the solvent in step (b) is any liquid substance capable of dissolving (+)-TBZ. Preferably, the solvent is selected from the group consisting of methanol, ethanol, and N-methyl-2-pyrrolidone.

In certain embodiments, the pH adjusting agent in step (c) is any agent that can adjust the solution to basic condition. Preferably, the pH adjusting agent is ammonium hydroxide, sodium hydroxide, or sodium carbonate, more preferably ammonium hydroxide.

In some embodiments, the crystal form is Form 2, and the method comprises:
a) obtaining a salt of (+)-TBZ from racemate TBZ using a resolving agent, preferably the resolving agent is (1S)-(+)-10-camphorsulfonic acid ((+)-CSA);
b) dissolving the salt of (+)-TBZ in a solvent to obtain a solution;
c) optionally adjusting the pH of the solution with a pH adjusting agent to basic condition, preferably to pH 7.5-8.5;
d) adding an anti-solvent to the solution of step (b); and
e) obtaining Form 2 by filtration.

In certain embodiments, the solvent in step (b) is any liquid substance capable of dissolving (+)-TBZ. Preferably, the solvent is selected from the group consisting of methanol, ethanol, and N-methyl-2-pyrrolidone.

In certain embodiments, the pH adjusting agent in step (c) is any agent that can adjust the solution to basic condition. Preferably, the pH adjusting agent is ammonium hydroxide, sodium hydroxide, or sodium carbonate, more preferably ammonium hydroxide.

In certain embodiments, the anti-solvent in step (d) is any liquid substance in which (+)-TBZ is poorly soluble. Preferably, the antisolvent is selected from the group consisting of water and polyvinylpyrrolidone.

The present application further provides a pharmaceutical composition comprising the crystal form of (+)-TBZ of the present application and at least one pharmaceutically acceptable excipient.

In some embodiments, the crystal forms of (+)-TBZ or the pharmaceutical formulation of the present disclosure can be formulated into a tablet, capsule, pill, granule, solution, suspension, syrup, injection (including injection solution, sterile powder for injection, and concentrated solution for injection), suppository, inhalant or spray.

In addition, the crystal forms of (+)-TBZ or the pharmaceutical composition of the present disclosure can also be administrated to a patient or subject in need of such treatment by any suitable administration mode, for example, oral, parenteral, rectal, intrapulmonary or topical administration. For the oral administration, the pharmaceutical composition may be formulated into an oral formulation, for example, an oral solid formulation such as a tablet, capsule, pill, granule and the like; or an oral liquid formulation such as an oral solution, oral suspension, syrup and the like. When formulated into an oral formulation, the pharmaceutical formulation may further comprise a suitable filler, binder, disintegrant, lubricant and the like. For parenteral administration, the pharmaceutical formulation may be formulated into an injection formulation including an injection solution, sterile powder for injection and concentrated solution for injection. When formulated into an injection formulation, the pharmaceutical composition may be produced by a conventional method in the pharmaceutical industry. When an injection is formulated, an additional agent may not be added to the pharmaceutical formulation, or a suitable additional agent may be added depending on the nature of the medicament. For rectal administration, the pharmaceutical formulation may be formulated into a suppository and the like. For intrapulmonary administration, the pharmaceutical formulation may be formulated into an inhalant or spray and the like.

In certain preferred embodiments, the crystal form of the present disclosure is present in the pharmaceutical composition or medicament in a therapeutically and/or prophylactically effective amount. In certain preferred embodiments, the crystal form of the present disclosure is present in the pharmaceutical composition or medicament in a unit dose.

The present application also provides a method of treating hyperkinetic movement disorders, comprising administering a therapeutically effective amount of the crystal form of (+)-TBZ or the pharmaceutical composition of the present application to a person suffering from a hyperkinetic movement disorder (e.g. Huntington's disease and tardive dyskinesia, TD).

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIGS. 1A-1B demonstrate XRD analysis of Form 1 of crystalline (+)-TBZ (free base) prepared by chiral resolution. FIG. 1A: original view; FIG. 1B: zoomed-in view.

FIGS. 2A-2B demonstrate XRD analysis of Form 2 of crystalline (+)-TBZ (free base) prepared by chiral resolution followed by anti-solvent addition. FIG. 2A: original view; FIG. 2B: zoomed-in view.

FIGS. 3A-3B demonstrate morphology of crystalline (+)-TBZ using de-ionized (DI) water as the anti-solvent. FIG. 3A: crystallization of (+) TBZ: 1.5 g/50 mL ethanol, anti-solvent: de-ionized (DI) water 50 mL, 1 mL/min; and FIG. 3B: crystallization of (+)-TBZ: 1.5 g/50 mL ethanol, anti-solvent: de-ionized (DI) water 100 mL, 1 mL/min.

FIG. 4 demonstrates morphology of crystalline (+)-TBZ using 1% PVP aqueous solution as the anti-solvent

FIGS. 5A-5C demonstrate morphology of crystalline (+)-TBZ prepared at different temperature. FIG. 5A: crystallization of (+)-TBZ at 30° C.: seeding: 1 g (+)-TBZ/50 mL ethanol; crystal growing: 10 g (+)-TBZ/50 mL NMP, 1 mL/min & anti-solvent: de-ionized (DI) water 50 mL, 1 mL/min; FIG. 5B: crystallization of (+)-TBZ at 5° C.: seeding: 400 mg (+)-TBZ/50 mL ethanol; crystal growing: 10 g (+)-TBZ/50 mL NMP, 1 mL/min & anti-solvent: de-ionized (DI) water 55 mL, 1 mL/min; and FIG. 5C: crystallization of (+)-TBZ at −5° C.: seeding: 300 mg (+)-TBZ/50 mL ethanol; crystal growing: 10 g (+)-TBZ/50 mL NMP, 1 mL/min & anti-solvent: de-ionized (DI) water 55 mL, 1 mL/min.

FIG. 6 demonstrates particle size distribution (PSD) of crystalline (+)-TBZ under various temperature control.

FIGS. 7A-7C demonstrate morphology and particle size distribution (PSD) of crystalline (+)-TBZ produced in scale-up process. FIG. 7A: 10-gram batch of crystalline (+)-TBZ (D10/D50/D90: 50.7/87.9/140.0 μm); FIG. 7B: 20-gram batch of crystalline (+)-TBZ (D10/D50/D90: 50.3/83.1/130.0 μm); and FIG. 7C: PSD of crystalline (+)-TBZ produced under scale-up process.

FIG. 8 demonstrates configuration of the cooling chamber.

FIG. 9 demonstrates microscopic image of crystalline (+)-TBZ obtained via cooling process at the scale of 3 grams with 200 rpm agitation.

FIG. 10 demonstrates microscopic image of crystalline (+)-TBZ obtained via cooling process at the scale of 12 grams with 200 rpm agitation.

FIG. 11 demonstrates microscopic image of crystalline (+)-TBZ obtained via cooling process at the scale of 12 grams batch with 500 rpm agitation.

FIGS. 12A-12C demonstrate differential scanning calorimetry (DSC) analysis of crystalline (+)-TBZ. FIG. 12A: crystalline (+)-TBZ, D50˜35 μm; FIG. 12B: crystalline (+)-TBZ, D50˜50 μm; and FIG. 12C: crystalline (+)-TBZ, rod-shaped.

FIGS. 13A-13C demonstrate thermogravimetric analysis (TGA) of crystalline (+)-TBZ. FIG. 13A: crystalline (+)-TBZ, D50˜35 μm; FIG. 13B: crystalline (+)-TBZ, D50˜50 μm; and FIG. 13C: crystalline (+)-TBZ, rod-shaped.

FIG. 14 demonstrates sustained release of (+)-TBZ from different polymeric formulations comprising crystalline (+)-TBZ.

FIG. 15 demonstrates sustained release of (+)-TBZ from different SAIB-based formulations comprising crystalline (+)-TBZ.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

The present application, therefore, provides methods of preparing (+)-TBZ in crystalline forms. Crystalline (+)-TBZ can be in the form of free base, salts, hydrates, or anhydrates, which have one or more desirable properties such as chemical purity, solubility, dissolution rate, crystal morphology, polymorphic stability, thermal stability, mechanical stability, storage stability, a low content of residual solvent, a low degree of hygroscopicity, and advantageous processing and handling characteristics such as flowability, wettability, compressibility and bulk density.

Preferably, crystalline (+)-TBZ is in the form of free base, where the nitrogen atoms are not protonated.

Different aspects of the invention are described below in further detail by embodiments, without being limited thereto. Each aspect of the invention may be described by one embodiment or by combining two or more of the embodiments.

### Definitions

It must be noted that as used herein and in the appended claims, the singular forms “a,” “an,” and “the” include plural reference unless the context clearly dictates otherwise.

As used herein, the term “about” preceding a numerical value or a series of numerical values means ±10% of the numerical value unless otherwise indicated. For example, “about 100 mg” means 90 to 110 mg.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure. As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “(+)-tetrabenazine or (+)-TBZ free base” refers to the solid form of “(+)-tetrabenazine or (+)-TBZ free base”, having a chemical structure, wherein the molecule is not associated with any acid molecule. As used herein, the term “(−)-tetrabenazine or (−)-TBZ free base” refers to the solid form of “(−)-tetrabenazine or (−)-TBZ free base”, having a chemical structure, wherein the molecule is not associated with any acid molecule. As used herein, the term “racemic mixture, racemic TBZ, racemate, TBZ racemate or racemate TBZ” refers to a mixture of (+)-TBZ and (−)-TBZ at about 1:1 ratio.

As used herein, the term “crystalline (+)-TBZ” is interchangeable with the term “(+)-TBZ crystal” and the term “crystallized (+)-TBZ” and the term “crystal form of (+)-TBZ” throughout this specification and the claims which follow, unless the context requires otherwise.

The term “hydrate” as used herein, refers to a crystalline solid where water is cooperated in or accommodated by the crystal structure [e.g. water is part of the crystal structure or entrapped into the crystal (water inclusions)]. Thereby, water can be present in a stoichiometric or non-stoichiometric amount.

The terms “physical form” and “solid form” are used interchangeably herein and refer to any crystalline and/or amorphous phase of a compound.

As used herein, X-ray diffraction [as known as powder X-ray diffraction (PXRD), X-ray powder diffraction (XRPD), or X-ray diffraction (XRD)] is a laboratory technique that reveals structural information, such as chemical composition, crystal structure, crystallite size, strain, preferred orientation and layer thickness. The XRD can be used to analyze a wide range of materials, from powder to solids such as thin films and nanomaterials. The peaks in an X-ray diffractogram are caused at certain diffraction angles (Bragg angles) by constructive interference from X-rays scattered by parallel planes of atoms in solid material, which are distributed in an ordered and repetitive pattern in a long-range positional order. Such a solid material is classified as crystalline material. A solid form of a compound that is not crystalline is defined as an amorphous material. An amorphous compound possesses no long-range order and does not display a definitive X-ray diffraction pattern. (see “Fundamentals of Powder Diffraction and Structural Characterization of Materials” by Vitalij et al., Kluwer Academic Publishers, 2003, page 3). A good X-ray diffractogram with clear, sharp peaks with low background noise should be obtained to enable data analysis and interpretation.

The term “2θ” or “2θ angle” or “2-Theta” used in the present application refers to the diffraction angle, and θ is the Bragg angle, and the unit of which is ° or degree. The error range of 2θ is between +0.1 and +0.5, preferably between +0.1 and +0.3, and can be −0.30, −0.29, −0.28, −0.27, −0.26, −0.25, −0.24, −0.23, −0.22, −0.21, −0.20, −0.19, −0.18, −0.17, −0.16, −0.15, −0.14, −0.13, −0.12, −0.11, −0.10, −0.09, −0.08, −0.07, −0.06, −0.05, −0.04, −0.03, −0.02, −0.01, 0.00, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, and more preferably +0.2.

As used herein, the term “substantially as shown” with reference to XRD means that variabilities in peak positions and relative intensities of the peaks are to be considered. For example, a typical precision of the 2-Theta values is in the range of +0.2° 2-Theta, preferably in the range of +0.1° 2-Theta. In addition, one skilled in the art will appreciate that relative peak intensities will show inter-apparatus variability as well as variability due to degree of crystallinity, preferred orientation, sample preparation and other factors known to those skilled in the art.

As used herein, the term “consisting essentially of” with reference to the amount of the crystalline (+)-TBZ in a composition means that slight variabilities in the amount are to be considered. This term is also to be understood herein in that such a composition comprises at least 96% by weight, preferably 98% by weight, more preferably 99% by weight, and most preferably 99.9% by weight of the crystalline (+)-TBZ as defined above, based on the total weight of the composition.

A crystalline solid form of (+)-TBZ may be referred to herein as being characterized by graphical data “as shown in” a figure. Such data include, for example, XRD, differential scanning calorimetry, and thermogravimetric analysis. The person skilled in the art understands that factors such as variations in instrument type, response and variations in sample directionality, sample concentration, sample purity, sample history and sample preparation may lead to variations for such data when presented in graphical form, for example variations relating to the exact peak positions and intensities. However, a comparison of the graphical data in the figures herein with the graphical data generated for an unknown physical form and the confirmation that two sets of graphical data relate to the same crystal form is well within the knowledge of a person skilled in the art. Multiple polymorph in a sample may also be determined by XRD. (see: US Patent Application No. 20190315744). All powder X-ray diffraction patterns were obtained by methods known in the art using a Bruker D2 Phaser XPRD analyzer A26-X1-A2B0B2A0 (Ser No.: 209872, Germany) with Cu anode.

Measurement of thermal analysis are conducted for the purpose of evaluating the physical and chemical changes that may take place in a heated sample. Thermal reactions can be endothermic (e.g., melting, boiling, sublimation, vaporization, desolvation, solid-solid phase transitions, chemical degradation, etc.) or exothermic (e.g., crystallization, oxidative decomposition, etc.) in nature. Such methodology has gained widespread use in the pharmaceutical industry in characterization of polymorphism. Thermal measurements have proven to be useful in the characterization of polymorphic systems. The most commonly applied techniques are thermogravimetry analysis (TGA), differential thermal analysis (DTA), and differential scanning calorimetry (DSC).

DSC is a thermodynamic tool for direct assessment of the heat energy uptake occurring in a sample within a controlled increase or decrease temperature process. The calorimetry throughout the process is applied to monitor the changes of phase transitions of the sample. DSC curves presented herein were obtained by methods known in the art using Waters Q200. The weight of the samples was about 1 to about 5 mg. The samples were scanned up from 25° C. to 200° C. at 5° C./min increment.

TGA is a measurement of the thermally induced weight loss of a material as a function of the applied temperature. TGA is restricted to transitions that involve either a gain or a loss of mass, and it is most commonly used to study desolvation processes and compound decomposition. TGA curves presented herein were obtained by methods known in the art using Waters TGA 550. The weight of the samples was about 5 to about 10 mg. The samples were scanned up from 25° C. to 650° C. at 10° C./min increment. Samples were purged with nitrogen gas at a flow rate of 25 mL/min. Samples were held in standard platinum pan covered with lids.

As used herein, the term “substantially as shown” with reference to DSC or TGA means that variabilities in peak positions, peak shape, heat absorbed or release, weight percentage loss, and/or curve shape are to be considered.

As used herein, a solvent is any liquid substance capable of dissolving (+)-TBZ. As used herein, the term “anti-solvent” means a liquid in which a compound is poorly soluble. The addition of an anti-solvent to a solvent reduces the solubility of a compound. As used herein a mixture of solvents refers to a composition comprising more than one solvent.

The starting material used in the method for preparing the crystal forms of the present disclosure may be (+)-TBZ or racemate TBZ in any form, and the specific forms include, but are not limited to, amorphous form, arbitrary crystal forms and the like.

According to the embodiments of the present application, (+)-TBZ can be prepared by chemical resolution of racemate TBZ using a resolving agent. Examples of the resolving agent include, but not limited to, (1S)-(+)-10-camphorsulfonic acid ((+)-CSA) as a resolving agent.

In some embodiments, crystalline (+)-TBZ is prepared by anti-solvent precipitation, solvent evaporation or temperature alteration. The different crystallization methods can be selected depending on the desired physiochemical properties of (+)-TBZ, or, on the subsequent processes for preparation of the pharmaceutical compositions. The present application enables practical, reproducible methods of the production of crystalline (+)-TBZ. Such methods can be easily scaled up under various temperature control, including at room temperature. This process is fast and only small amount of solvent is required while the yield can be over 80%. More preferably, the yield can be over 90%.

Furthermore, the present application demonstrates successful crystal particle size control over different batch sizes. Since particle size of an active pharmaceutical ingredient (API) plays vital role in drug release, such API crystallization process invented herein with easy, tunable API particle size control would provide tremendous advantages on formulation development in controlling release profile via API particle size selection. Such tunable size control can be achieved by, but not limited to temperature control, feeding rate of API stock solution/anti-solvent and the ratio of solvent/anti-solvent.

In one embodiment, (+)-TBZ provides higher or comparable amount of therapeutically active VMAT2 inhibitors [(+)-TBZ and (+)-DHTBZ] as TBZ racemate in rat liver microsomes, while the use of (+)-TBZ avoids the generation of any undesired metabolites [e.g. isoforms of (−)-DHTBZs] which can potentially lead to significant side effects through off-targeting binding to serotonin (5-HT1A, 5-HT2A, 5-HT2B) or dopamine (D1 or D2) receptors [Harriott et al. “VMAT2 Inhibitors and the Path to Ingrezza (Valbenazine)”, Progress in Medicinal Chemistry, Volume 57, page 87-111 (2018)].

In some embodiments, the shape and size of crystalline (+)-TBZ is tailored using the methods in the present application. The corresponding physiochemical properties of crystallized (+)-TBZ with defined morphology (shape and size) can dictate the quality of the final pharmaceutical compositions (e.g., improved API flowability). In some embodiments, the present application discloses methods of manufacturing crystalline (+)-TBZ at controllable batch sizes, crystal shape and particle size distribution all in a reproducible manner with over 80% yield. In still another embodiment, crystalline (+)-TBZ obtained under the methods provided herein demonstrates very good flowability, which are of practical use for drug product development (e.g., ease of filling and handling).

In one embodiment, a pharmaceutical composition comprises crystalline (+)-TBZ, a biocompatible solvent, and a biodegradable polymer selected from the group consisting of a homopolymer polylactide or polylatic acid (PLA), a copolymer poly (lactic acid-co-glycolic acid) or poly (lactide-co-glycolide) (PLGA), and a combination thereof. The biodegradable polymers have a weight average molecular weight (Mw) ranging from about 1,000 to about 120,000, preferably from about 5,000 to about 40,000, as determined by gel permeation chromatography (GPC). The biodegradable polymer can have one ester terminal functional group and one hydroxyl end group and can also be made to have one or two carboxyl terminal groups. The biodegradable polymer can be dissolved with biocompatible solvent selected from the group consisting of N-methyl-2-pyrrolidone, 2-pyrrolidone, N,N-dimethylformamide, dimethyl sulfoxide, benzyl alcohol, benzyl benzoate, triacetin, and combinations thereof to make polymeric solutions as the delivery vehicles for therapeutically active VMAT2 inhibitors.

In one embodiment, a pharmaceutical composition comprises crystalline (+)-TBZ, a biocompatible solvent, and a biodegradable, hydrophobic, non-polymeric material. The biodegradable, hydrophobic, non-polymeric material is sucrose acetate isobutyrate (SAIB). SAIB is the product of esterification of disaccharide, sucrose, with two acetic and six isobutyric acid. It is extremely viscous with viscosity of over 100,000 cPs and is essentially insoluble in aqueous mediums. SAIB can be heated up to significantly reduce its viscosity, followed by mixing with a biocompatible solvent selected from the group consisting of ethanol, N-methyl-2-pyrrolidone, 2-pyrrolidone, N,N-dimethylformamide, dimethyl sulfoxide, benzyl alcohol, benzyl benzoate, triacetin, and combinations thereof to make low viscous solutions as the delivery vehicles for therapeutically active VMAT2 inhibitors.

In a further embodiment, the crystallinity of (+)-TBZ allows one to distinguish the formation of an enantiomer from a racemate which is impossible to be certain even by NMR. In a further embodiment, the crystallinity of (+)-TBZ described in this invention is unique and can be obviously distinguished from that of TBZ racemate (WO 2012/081031 A1) by a person having ordinary skill in the art.

### EXAMPLES

The following examples of the present application are to further illustrate the nature of the application. It should be understood that the following examples do not limit the application and the scope of the application is to be determined by the appended claims.

### Abbreviations

Unless indicated otherwise, the abbreviations for chemical reagents and synthesis conditions have their ordinary meaning known in the art as follows:
“EA” refers to ethyl acetate;
“PE” refers to petroleum ether;
“r.t.” and “rt” and “RT” refer to room temperature;
“THF” refers to tetrahydrofuran;
“DCM” refers to dichloromethane;
“Hex” refers to hexanes;
“DMF” refers to dimethylformamide;
“NMP” refers to N-methyl-2-pyrrolidone;
“PVP” refers to polyvinylpyrrolidone;
“h” refers to hours;
“min” refers to minutes;
“MeOH” refers to methanol.
“EtOH” refers to ethanol.
“i-PrOH” refers to isopropanol.

### Example 1: Preparation of (+)-TBZ

(+)-Tetrabenazine was prepared according to the procedure disclosed in CN110092785A. (±)-Tetrabenazine (3 g, 9.45 mmol, 1 eq) and (1S)-(+)-1-camphorsulfonic acid (4.39 g, 18.9 mmol, 2.0 eq) were added in 30 mL of ethyl acetate (10 V, v/w), and the mixture was refluxed for 96 hours. The solution was cooled to room temperature and stirred at RT, followed by temperature reduction to 10° C. with stirring for 0.5 h. Surprisingly, there was no white solid precipitation as disclosed in CN110092785A. The aforementioned mixture was further cooled down to 0° C. After stirring at 0° C. for 8 hours, there was still no solid precipitation, thus, the mixture was warmed to room temperature (RT) and stirred for extra 2 days till solid was precipitated. The precipitated product (+)-Tetrabenazine-(+)-CSA salt was filtered and washed with 6 mL ethyl acetate (2V, v/w). The yield of (+)-Tetrabenazine-(+)-CSA salt was 24.9% with chiral purity of 36.32%, vs. 79.1% and 99.5% disclosed in CN11009278SA, respectively. This is quite unexpected because the yield of the (+)-Tetrabenazine and chiral purity were much lower than those disclosed in CN110092785A. The procedure was repeated and the observation was confirmed

### Example 2. Crystalline (+)-TBZ Preparation: Chiral Resolution I

20 g of (3R,11bR)- and (3S,11bS)-tetrabenazine or TBZ racemate and at least 0.1 equivalent molar of (1S)-(+)-10-CSA were dissolved in warm acetone and stirred for 1 h. Ethyl acetate was added and stirred for 48 hours at RT to obtain (+)-TBZ-(+)-CSA salt. The obtained (+)-TBZ-(+)-CSA salt was dissolved in methanol. Aqueous solution of ammonium hydroxide was then added to adjust pH to 7.5-8.5 and then to allow crystallization of (+)-TBZ (free base). The obtained crystalline (+)-TBZ (free base) was then filtered and washed with water to give about 4.5 g colorless solid. The obtained (3R,11bR)-tetrabenazine or crystalline (+)-TBZ was then dried at 40° C. Final purity was over 99% and yield was 30%. XRD were obtained using Bruker D2 Phaser XPRD analyzer A26-X1-A2B0B2A0 (Ser No.: 209872, Germany) with Cu anode. Divergence and anti-scatter slit were set as 0.2 mm and 1.0 mm, respectively. About 10 mg of the sample was scanned from 3° to 45° at a step size of 0.02° per second. The parameters were tabulated in Table 1.

TABLE 1
Parameters of X-ray powder diffractometry

| Anode | Cu | |
|---|---|---|
| Divergence slit | 0.2 | mm |
| Anti-scatter slit | 1.0 | mm |
| Sample | 10 | mg |
| Scan Range | 3°~45° | |
| Step size | 0.02° | |
| Time per Step | 0.5 | sec |

| | |
|---|---|
| Temperature | Room temp. |

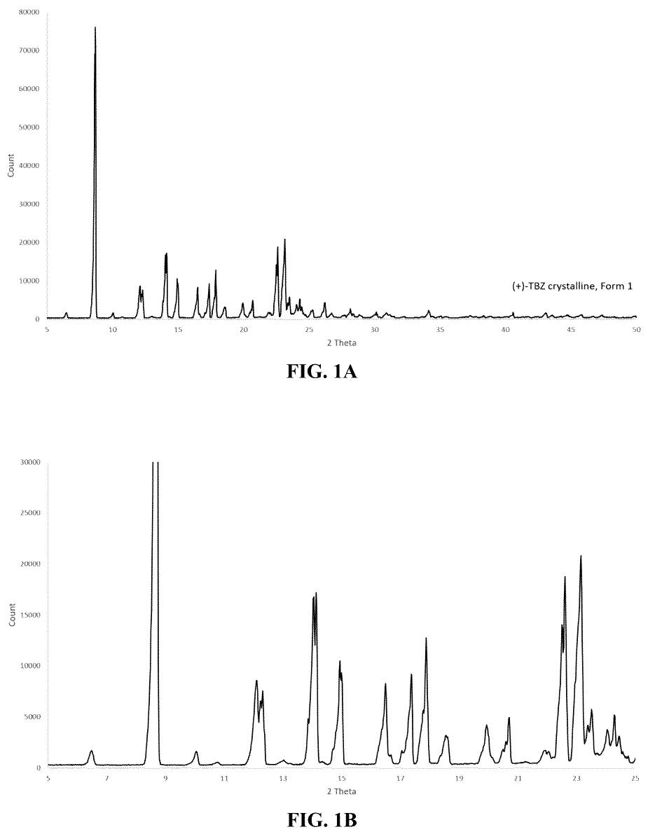

FIGS. 1A (original view) and 1B (zoomed-in view) showed the XRD pattern of crystalline (+)-TBZ obtained using chiral resolution method (Crystalline (+)-TBZ (free base), Form 1). Characteristic peaks of Form 1 of crystalline (+)-TBZ are summarized in Table 2.

TABLE 2
List of Characteristic Peaks: Crystalline (+)-TBZ, Form 1

| Diffraction 2θ Angle (°) | Relative intensity |
|---|---|
| 6.48 | 2.2 |
| 8.70 | 100.0 |
| 12.09 | 11.3 |
| 14.05 | 22.1 |
| 14.13 | 22.6 |
| 14.94 | 13.8 |
| 15.00 | 12.3 |
| 16.50 | 10.9 |
| 17.37 | 12.1 |
| 17.89 | 16.8 |
| 18.56 | 4.2 |
| 19.94 | 5.5 |
| 20.71 | 6.5 |
| 21.96 | 2.2 |
| 22.51 | 18.5 |
| 22.61 | 24.7 |
| 23.16 | 27.4 |
| 23.52 | 7.6 |
| 24.29 | 6.8 |
| 24.76 | 1.5 |
| 25.16 | 2.6 |
| 26.19 | 5.5 |
| 26.22 | 5.6 |
| 31.09 | 1.4 |
| 32.31 | 0.8 |
| 39.92 | 0.9 |

### Example 3. Crystalline (+)-TBZ Preparation: Chiral Resolution II

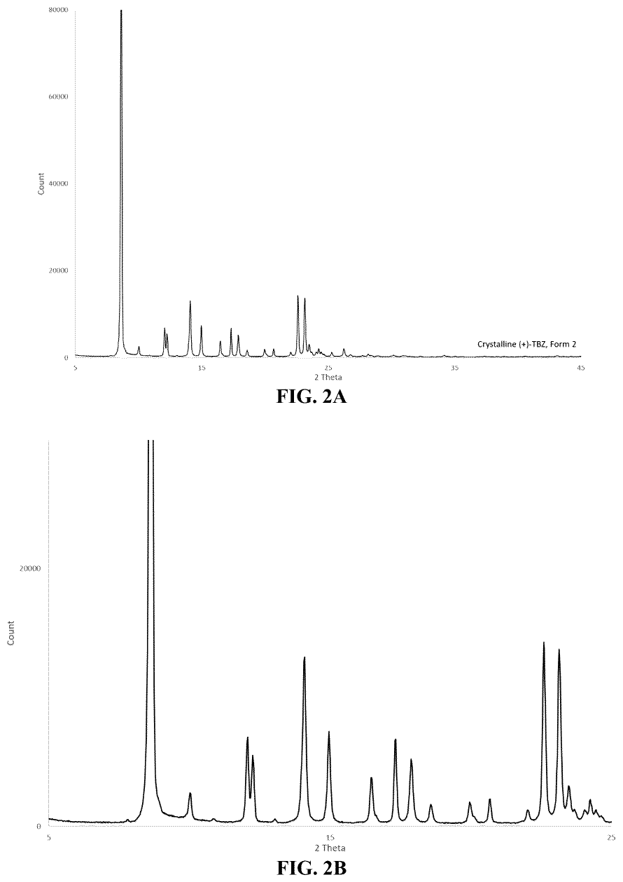

5 g of (3R,11bR)- and (3S,11bS)-tetrabenazine or TBZ racemate and at least 0.1 equivalent molar of (1S)-(+)-10-CSA were dissolved in a co-solvent of acetone and ethyl acetate, followed by stirring for 48 hours at RT to obtain (+)-TBZ-(+)-CSA salt. The obtained (+)-TBZ-(+)-CSA salt was dissolved in methanol. A pH adjusting agent, ammonium hydroxide, was then added to adjust pH to 7.5-8.5. Other pH adjusting agents can also be used herein, and examples of such agents include, but not limited to ammonium hydroxide, sodium hydroxide, and sodium carbonate. Water was introduced to allow the crystallization of (+)-TBZ to obtain crystalline (+)-TBZ (free base). Final purity was over 99%. XRD analyses were obtained using Bruker D2 Phaser XPRD analyzer A26-X1-A2B0B2A0 (Ser No.: 209872, Germany) with Cu anode. Divergence and anti-scatter slit were set as 0.2 mm and 1.0 mm, respectively. About 10 mg of the sample was scanned from 3° to 45° at a step size of 0.02° per second. The parameters were the same as tabulated in Table 1. FIGS. 2A (original view) and 2B (zoomed-in view) demonstrated XRD pattern of Form 2 of crystalline (+)-TBZ obtained via chiral resolution II (Crystalline (+)-TBZ (free base), Form 2). Characteristic peaks of Form 2 of crystalline (+)-TBZ are summarized in Table 3. It is unexpected that at least eleven significant peaks were missing at 2 theta angle 6.48°, 14.139, 15.00°, 21.96°, 22.51°, 24.76°, 25.16°, 26.199, 31.09°, 32.31°, and 39.92°, compared to crystalline (+)-TBZ Form 1 disclosed in Example 2. Crystalline (+)-TBZ (free base, Form 2) obtained in Example 3 was used as the raw material for the subsequent Examples in the present application.

TABLE 3
List of Characteristic Peaks: Crystalline (+)-TBZ, Form 2

| Diffraction 2θ Angle (°) | Relative intensity (%) |
|---|---|
| 8.64 | 100.0 |
| 12.06 | 6.2 |
| 14.09 | 12.2 |
| 14.96 | 6.7 |
| 16.47 | 3.3 |
| 17.32 | 6.1 |
| 17.89 | 4.6 |
| 18.59 | 1.3 |
| 19.98 | 1.5 |
| 20.69 | 1.7 |
| 22.60 | 13.2 |
| 23.15 | 12.6 |
| 23.49 | 2.6 |
| 24.26 | 1.6 |
| 26.24 | 1.7 |

### Example 4. Crystallization Method I: Crystallization by Anti-Solvent Addition

Inventors of the present application have found that crystallization of (+)-TBZ can be achieved by dropwise dosing of an anti-solvent into a (+)-TBZ stock solution. Crystal morphology and size can be affected by several factors, including concentration of (+)-TBZ stock solution, types of solvent and anti-solvent, dosing rate, solvent to anti-solvent ratio, agitation speed, temperature, and the employment of seeding procedure.

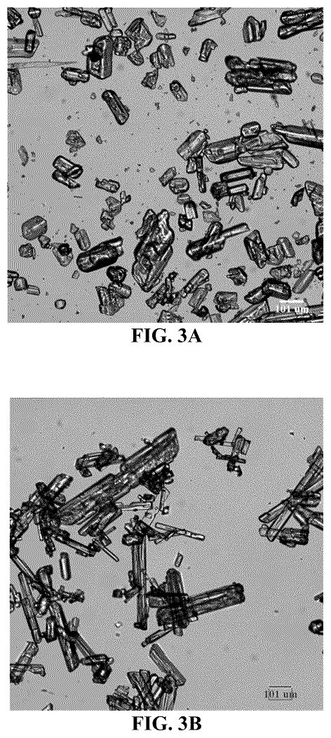

In this example, 1.5 g of (+)-TBZ was dissolved in 50 mL ethanol to form a clear solution. Then 50 mL of de-ionized (DI) water, as the anti-solvent, was introduced dropwise into the solution at 1 mL/min with agitation using an overhead stirrer at 800 rpm (IKA, EUROSTAR 40 DIGITAL) while temperature was maintained at 30° C. Under this condition, shorter rod-shaped crystals were produced (FIG. 3A), whereas by continuously adding twice the amount of water into the same solution, the crystals transferred to much longer rods with even larger aspect ratio (FIG. 3B). XRD were obtained using Bruker D2 Phaser XPRD analyzer A26-XI-A2B0B2A0 (Ser No.: 209872, Germany) with Cu anode. Divergence and anti-scatter slit were set as 0.2 mm and 1.0 mm, respectively, and the XRD confirmed that the crystals obtained in this Example are Form 2

### Example 5. Crystallization Method II: Anti-Solvent Addition with Polyvinylpyrrolidone

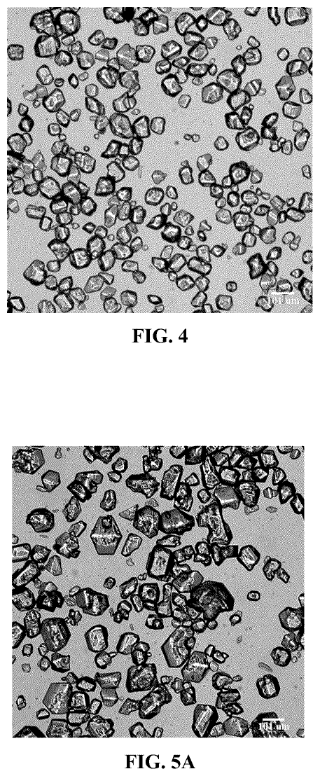

1.375 g (+)-TBZ was dissolved in 50 mL ethanol at 30° C. in a water bath with the aid of overhead stirrer set at 800 rpm (IKA, EUROSTAR 40 DIGITAL). After (+)-TBZ ethanol solution was made and temperature was stabilized at 30° C., 30 mL of 1% polyvinylpyrrolidone (PVP) aqueous solution (anti-solvent) was added at the rate of 1 mL/min dropwise (via a syringe pump) with stirring at 800 rpm. Each drop of anti-solvent created local over-saturation leading to nucleation but immediate re-solubilized to become clear. After 30 mL of anti-solvent addition, the solution was kept stirring at 800 rpm for 30 min at 30° C. During this process, the solution was clear without any visible nucleus. Subsequently, 10 mL anti-solvent was again dosed at the rate of 1 mL/min dropwise with stirring at 800 rpm at 30° C. During this dosing process, the formed nucleus re-solubilized slower and the stable (+)-TBZ seeds were eventually formed. The seeded (+)-TBZ suspension was allowed to grow for another 60 min with stirring at 30° C. In the meantime, the nucleation and crystal growth increasingly accelerated. After this crystal growing stage, 90 mL of anti-solvent was again added in three portions (30 mL each) at the rate of 1 mL/min (dropwise) with stirring at 800 rpm at 30° C. After each portion was introduced, the solution was stirred continuously for 30 min to allow crystal growing before adding another portion of the anti-solvent. After the last 30 min of crystal growing stage, crystallization was completed and the crystals were collected by filtration and drying (37° C.). The yield was around 90%. Crystals produced via this approach were much easier to be dispersed (FIG. 4) and were confirmed by XRD to be Form 2. This process was robust and reproducible with D10/D50/D90 about 42.2/71.9/116 μm (Malvern, Mastersizer 3000E).

### Example 6. Crystallization Method III: (+)-TBZ Crystalline Size Control

The present application also enables the production of crystalline (+)-TBZ in various size ranges in a controllable manner. Tunable parameters for size control over crystalline (+)-TBZ production includes but not limited to, control over process temperature, control over feeding rate of the API stock solution/anti-solvent, and control over the ratio of solvent/anti-solvent. For example, this can be achieved by controlling the crystallization at a specific temperature. An in-line two-step process comprising of first, the seeding stage, followed by second, the nucleation/crystal growing stage was introduced.

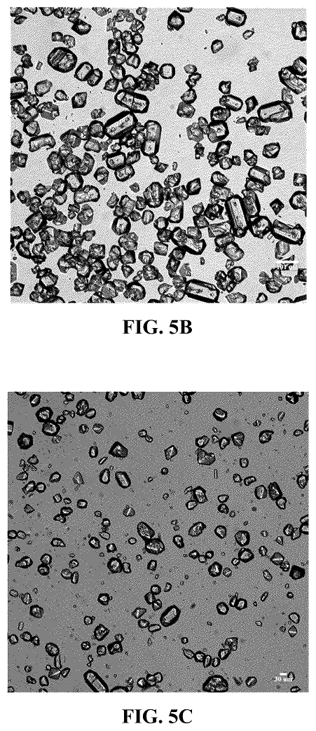

In one example, the seeding process was accomplished by dissolving 1 g of (+)-TBZ in 50 mL ethanol as the first (+)-TBZ stock solution where 40 mL de-ionized (DI) water (as the anti-solvent) was dropwise added at 1 mL/min rate with 300 rpm agitation (IKA, EUROSTAR 40 DIGITAL) while the whole process was controlled at 30° C. Meanwhile, to continue the process for nucleation/crystal growing stage, the second (+)-TBZ stock solution was prepared by dissolving 10 g of (+)-TBZ in 50 mL of N-methyl-2-pyrrolidone (NMP). Following the seeding process (after adding the first API stock solution), further nucleation and crystal growing took place while introducing 50 mL of the second (+)-TBZ stock solution and 50 mL of de-ionized (DI) water simultaneously into the seeding solution at 1 mL/min rate with 300 rpm agitation (IKA, EUROSTAR 40 DIGITAL). The whole process was controlled at 30° C. After crystallization process was completed, the obtained crystals were washed several times with de-ionized (DI) water and then collected by filtration through 0.2 μm filter cup, followed by oven drying at 37° C. overnight. The yield was around 75-80%. Crystals produced via this approach were easy to be dispersed. The whole process was robust, reproducible and can be carried out in less than 2 hours (FIG. 5A).

In another example, the system temperature was lowered to 5° C. with some adjustment on the seeding process. 400 mg of (+)-TBZ was dissolved in 50 mL ethanol as the first API stock solution. 30 mL of de-ionized (DI) water (as anti-solvent) was added dropwise into the first API stock solution at 1 mL/min rate with 300 rpm agitation (IKA, EUROSTAR 40 DIGITAL). The whole process was controlled at 5° C. Meanwhile, the second API stock solution was prepared by dissolving 10 g of (+)-TBZ in 50 mL of NMP. Following the seeding process, further nucleation and crystal growth took place after 55 mL of the second (+)-TBZ stock solution and additional 55 mL of de-ionized (DI) water were dosed simultaneously into the seeding solution at the rate of 1 mL/min with 300 rpm agitation (IKA, EUROSTAR 40 DIGITAL). The whole process was also controlled at 5° C. After crystallization process was completed, the obtained crystals were washed several times with de-ionized (DI) water and then collected by filtration through 0.2 μm filter cup, followed by oven drying at 37° C. overnight. Smaller crystals with yield about 85% (FIG. 5B) were generated.

In still another example, the whole process temperature was further lowered to −5° C. with the initial amount of (+)-TBZ reduced to 300 mg for the seeding process, while the parameters for the following nucleation/crystal growing process kept the same. Even smaller crystals with yield about 85% could be produced (FIG. 5C).

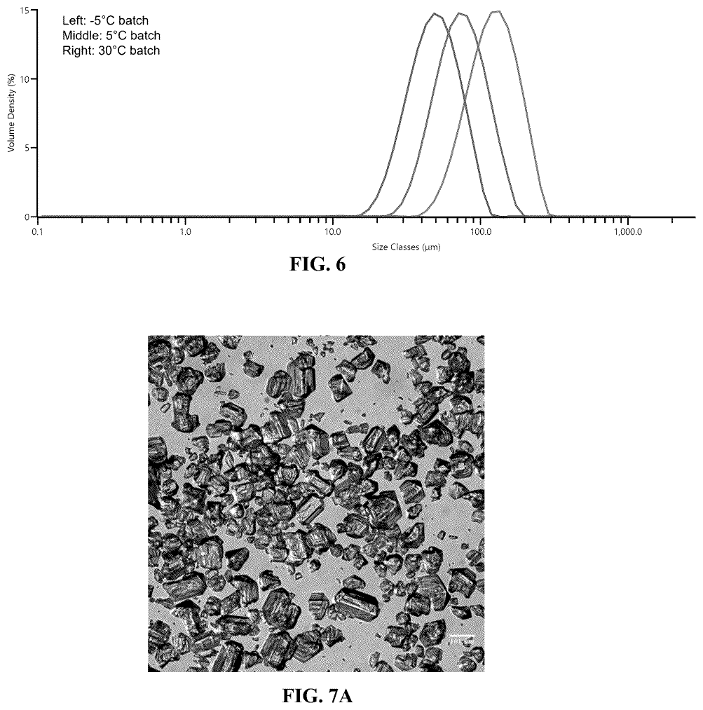

XRD confirmed that all the crystals obtained in this Example are Form 2. Particle size analysis results of the Form 2 (+)-TBZ crystalline prepared under different temperature condition using Malvern Mastersizer 3000 were summarized in FIG. 6 and Table 4.

TABLE 4
Summary of PSD of Form 2 (+)-TBZ crystalline
prepared under various temperature

| Sample | D10 (μm) | D50 (μm) | D90 (μm) | Span |
|---|---|---|---|---|
| (+)-TBZ crystalline, 30° C. | 28.6 | 48.5 | 78.7 | 1.032 |
| (+)-TBZ crystalline, 5° C. | 43.8 | 73.5 | 121.0 | 1.050 |
| (+)-TBZ crystalline, −5° C. | 72.2 | 123.0 | 197.0 | 1.015 |

### Example 7. Crystallization Method IV: Process Scale-Up of Anti-Solvent Addition

The present application also enables the scale-up production (+)-TBZ crystalline with identical particle size distribution in a controllable manner.

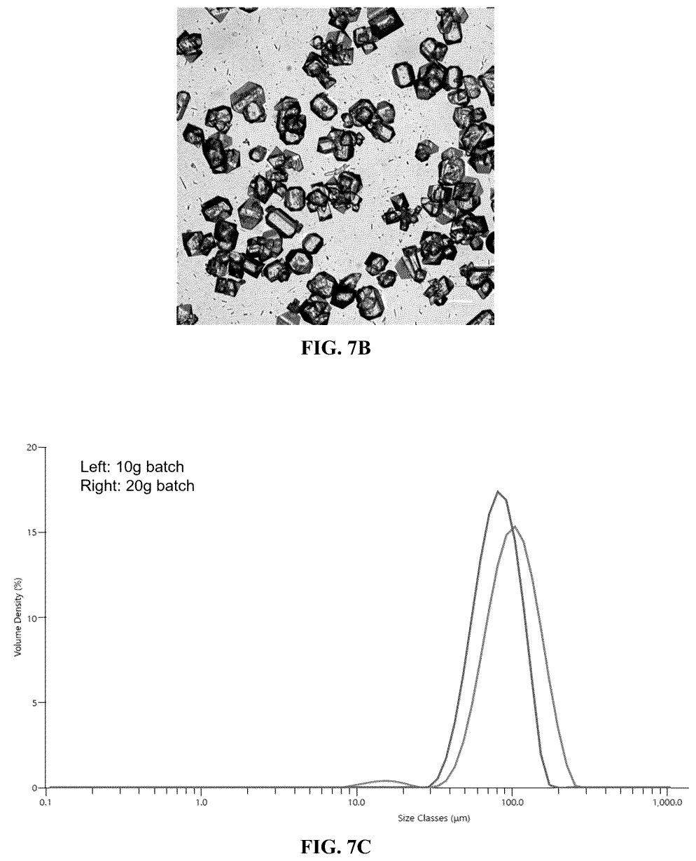

In one example, a production of 10-gram batch was initiated as the first seeding stage was controlled at 20° C. with 300 rpm agitation. This was carried out by dissolve 200 mg of (+)-TBZ in 50 mL of EtOH as the first API stock solution. Next, 30 mL de-ionized (DI) water was filled into a polypropylene syringe (anti-solvent) and was dropwise feed into the first API stock solution via a syringe pump at a rate of 1 mL/min. In the second stage, nucleation and crystal growing (controlled at 20° C., 300 rpm agitation) were proceeded by dissolving 10 g (+)-TBZ in 50 mL NMP as the second API stock solution. Next, about 110 mL de-ionized (DI) water was filled into another polypropylene syringe as the anti-solvent, followed by simultaneously feeding the second API stock solution and the anti-solvent via syringe pumps at a rate of 1 mL/min and 2 mL/min, respectively. After finish, the crystals were washed with 1 L de-ionized (DI) water and then collected via filtration (0.45 μm Nylon filter), followed by oven drying at 40° C. overnight. Crystals were investigated via both Malvern particle sizer and a light microscope to confirm particle shape/size and size distribution (D10/D50/D90: 50/85/140 μm) (FIGS. 7A and 7C).

In another example, a production of 20-gram batch was initiated as the first seeding stage was controlled at 10° C. with 300 rpm agitation. This was carried out by dissolving 800 mg of (+)-TBZ in 100 mL of EtOH as the first API stock solution. Next, 60 mL de-ionized (DI) water was filled into a polypropylene syringe (anti-solvent) and was dropwise feed into the first API stock solution via a syringe pump at a rate of 2.5 mL/min. In the second stage of nucleation and crystal growing, system temperature was kept the same at 10° C. with 300 rpm agitation, followed by dissolving 20 g (+)-TBZ in 100 mL NMP as the second API stock solution. Next, about 110 mL de-ionized (DI) water was filled into another polypropylene syringe as the anti-solvent, followed by simultaneously feeding the second API stock solution and the anti-solvent via syringe pumps at a rate of 2.5 mL/min. After finish, the crystals were washed with 1 L de-ionized (DI) water and then collected via filtration (0.45 μm Nylon filter), followed by oven drying at 40° C. overnight. The obtained crystalline (+)-TBZ was investigated via both Malvern particle sizer and a light microscope to confirm particle shape/size (FIG. 7B) and size distribution (D10/D50/D90: 50/85/130 μm, FIG. 7C). XRD confirmed that all the crystals obtained in this Example are Form 2.

### Example 8. Crystallization Method V: Crystallization Via Cooling: 3 Grams Batch

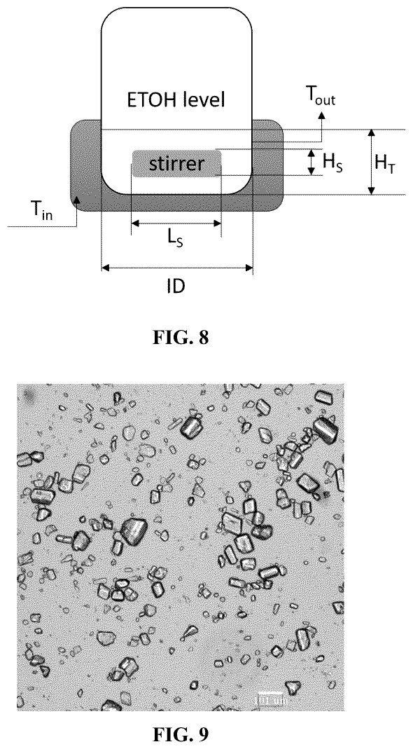

3 grams of (+)-TBZ was dissolved in 20 mL ethanol at about 63° C. with moderate mixing in a 120 mL glass vial. Once the solution turned clear and solid particulate was absent, the temperature of the solution was cooled at a speed of ˜2° C./min by coupling a glass double-layer jacket and a water-circulation chiller. The solution was kept agitating at 200 rpm. White precipitates began to be observed at about 42˜45° C. and the suspension was maintained at this temperature for 1 hr. After this isothermal interval, the temperature of (+)-TBZ suspension was decreased at a speed of 10° C./50 min, followed by a temperature gradient of 4° C./10 min to 5° C. Before harvesting, (+)-TBZ suspension was equilibrated at 5° C. for 2 hours, followed by filtration and drying at room temperature. The yield was ˜80%. The configuration of cooling chamber is plotted in FIG. 8. Operation parameters summarized in Table 5 and FIG. 8 were simply to demonstrate preparation of crystalline (+)-TBZ via the cooling method; a person with ordinary skill in the art should be able to obtain desired crystalline (+)-TBZ by following or modifying the setup and parameters accordingly. Morphology of crystalline (+)-TBZ was confirmed under microscope (FIG. 9), while PSD of the crystalline (+)-TBZ was characterized via Malvern Master Sizer 3000E (Table 6). XRD confirmed that the crystals obtained in this Example are Form 2.

TABLE 5
Operation parameters of 3 g (+)-TBZ cooling crystallization

| | | | | Inner | |
|---|---|---|---|---|---|
| Volume | Size | Total | Height of | diameter | Length of |
| of ETOH | of Stir | height, | Stir bar, | of chamber, | Stir bar, |
| (mL) | bar (cm³) | H_{T} (cm) | H_{S} (cm) | ID (cm) | L_{S} (cm) |
| 20 | 1.8 | 1.4 | 0.8 | 5 | 4 |

TABLE 6
PSD of Form 2 crystalline (+)-TBZ obtained via cooling process

| crystalline (+)-TBZ | D₁₀ (um) | D₅₀ (um) | D₉₀ (um) | Span |
|---|---|---|---|---|
| 3 g-01 | 17.3 | 38.5 | 75 | 1.5 |
| 3 g-02 | 13.3 | 27.9 | 51 | 1.4 |
| 3 g-03 | 23.2 | 53.6 | 102 | 1.5 |
| 3 g-04 | 16.8 | 34.4 | 61.4 | 1.3 |
| 3 g-05 | 16.8 | 33.8 | 60.4 | 1.3 |
| 3 g-06 | 26.3 | 56.6 | 109 | 1.5 |
| 3 g-07 | 22 | 45.9 | 85.7 | 1.4 |
| 3 g-08 | 16.6 | 33.7 | 60.7 | 1.3 |

### Example 9. Crystallization Method VI: Crystallization Via Cooling: 12 Grams Batch

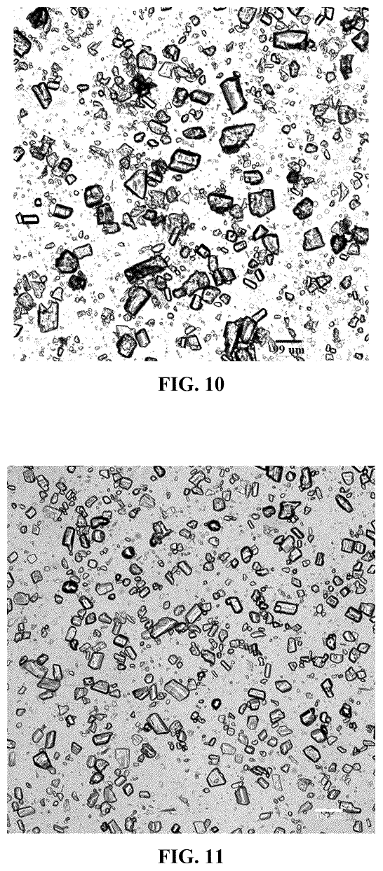

12 grams of TBZ was dissolved in 80 mL of heated ethanol at about 63° C. with moderate mixing in a 120 mL glass vial. Once the solution turned clear and solid particulate was absent, the temperature of the solution was cooled at a speed of ˜2° C./min by coupling a glass double-layer jacket and a water-circulation chiller. The solution was kept agitating at 200 rpm. White precipitates began to be observed at about 42˜45° C. and the suspension was maintained at this temperature for 1 hr. After this isothermal interval, the temperature of (+)-TBZ suspension was decreased at a speed of 10° C./50 min, followed by a temperature gradient of 4° C./10 min to 5° C. Before harvesting, (+)-TBZ suspension was equilibrated at 5° C. for 2 hours, followed by filtration and drying at room temperature. The yield was ˜90%. The configuration of cooling chamber was the same as plotted in FIG. 8. Operation parameters were summarized in Table 7. Morphology of crystalline (+)-TBZ was confirmed under microscope (FIG. 10), while PSD of the crystalline (+)-TBZ was characterized via Malvern Master Sizer 3000E (Table 8). XRD confirmed that the crystals obtained in this Example are Form 2.

TABLE 7
Operation parameters of 12-g (+)-TBZ
cooling crystallization agitated at 200 rpm

| | | | | Inner | |
|---|---|---|---|---|---|
| Volume | Size | Total | Height of | diameter | Length of |
| of ETOH | of Stir | height, | Stir bar, | of chamber, | Stir bar, |
| (mL) | bar (cm³) | HT (cm) | HS (cm) | ID (cm) | LS (cm) |
| 80 | 1.8 | 5.1 | 0.8 | 5 | 4 |

TABLE 8
PSD of Form 2 crystalline (+)-TBZ obtained via cooling
process at the scale of 12 grams with 200 rpm agitation

| crystalline (+)-TBZ | D10 (um) | D50 (um) | D90 (um) | Span |
|---|---|---|---|---|
| 12 g-01 | 20.2 | 51.4 | 103.8 | 1.6 |
| 12 g-02 | 24.2 | 61.1 | 120.5 | 1.6 |
| 12 g-03 | 18.4 | 57.9 | 125.7 | 1.9 |

### Example 10. Crystallization Method VII: Crystallization Via Cooling at Different Mixing Speed

12 grams of (+)-TBZ was dissolved in 80 mL in heated ethanol at about 63° C. with moderate mixing in a 120 mL glass vial. Once the solution turned clear and solid particulate was absent, the temperature of the solution was cooled at a speed of ˜2° C./min by coupling a glass double-layer jacket and a water-circulation chiller. The solution was kept agitating at 500 rpm. White precipitates began to be observed at about 42˜45° C. and the suspension was maintained at this temperature for 1 hr. After this isothermal interval, the temperature of (+)-TBZ suspension was decreased at a speed of 0.2° C./min, followed by a temperature gradient of 0.4° C./min to 5° C. Before harvesting, (+)-TBZ suspension was equilibrated at 5° C. for 2 hours, followed by filtration and drying at room temperature. The yield is ˜90%. The configuration of cooling chamber was the same as plotted in FIG. 8. Operation parameters were summarized in Table 9. Morphology of crystalline (+)-TBZ was confirmed under microscope (FIG. 11), while PSD of the crystalline (+)-TBZ was characterized via Malvern Master Sizer 3000E (Table 10). XRD confirmed that the crystals obtained in this Example are Form 2.

TABLE 9
Operation parameter of 12-g (+)-TBZ
crystallization: agitated at 500 rpm

| | | | | Inner | |
|---|---|---|---|---|---|
| Volume | Size | Total | Height of | diameter | Length of |
| of ETOH | of Stir | height, | Stir bar, | of chamber, | Stir bar, |
| (mL) | bar (cm³) | HT (cm) | HS (cm) | ID (cm) | LS (cm) |
| 80 | 1.8 | 5.1 | 0.8 | 5 | 4 |

TABLE 10
PSDs of Form 2 crystalline (+)-TBZ obtained via cooling
process at the scale of 12 grams with 500 rpm agitation

| crystalline (+)-TBZ | D10 (um) | D50 (um) | D90 (um) | Span |
|---|---|---|---|---|
| 12 g-04 | 15.9 | 34.9 | 65.8 | 1.4 |
| 12 g-05 | 17.7 | 40.8 | 80.4 | 1.5 |
| 12 g-06 | 16.7 | 38 | 76 | 1.6 |

### Example 11. Crystallization of (+)-TBZ: Improved Flowability

The present application enables the production of crystalline (+)-TBZ in various particle size ranges and crystal morphology (e.g. rod-shaped crystals and more symmetric crystals). As API powder/crystalline flowability can be critical in the manufacture of a drug product, flow property of crystalline (+)-TBZ in the present application was investigated. USP characterized flowability category is shown in Table 11.

TABLE 11
USP 1174 Powder Flow

| Flow Property | Angle of Repose (°) |
|---|---|
| Excellent | 25-30 |
| Good | 31-35 |
| Fair (aid not needed) | 36-40 |
| Passable (may hang up) | 41-45 |
| Poor (must agitate, vibrate) | 46-55 |
| Very poor | 56-65 |
| Very, very poor | >66 |

Most of the Form 2 crystalline (+)-TBZ produced in the present application showed good flow property, while rod-shaped crystals showed poorer flowability (Table 12). It was unexpected that with the same D50 value of about 80 μm, crystalline morphology leads to significant difference on powder followability (angle of repose was about 35° and about 64° for more symmetric crystalline (+)-TBZ and rod-shaped crystalline (+)-TBZ, respectively). The crystallization method developed in the present application allows manufacturing crystalline (+)-TBZ particles with good flow property in wide range of particle sizes.

TABLE 12
Flow property of Form 2 crystalline (+)-TBZ

| Crystalline (+)-TBZ | Response |
|---|---|
| with different D50 | Angle (°) |
| 15 μm | 45.3 ± 5.0 |
| 35 μm | 43.3 ± 2.5 |
| 50 μm | 36.7 ± 7.6 |
| 80 μm | 34.7 ± 1.2 |
| 125 μm | 35.3 ± 2.3 |
| Rod shape (80 μm) | 64.3 ± 1.2 |

### Example 12. Crystallization of (+)-TBZ with Tailored Size Control: Differential Scanning Calorimetry (DSC) Analysis

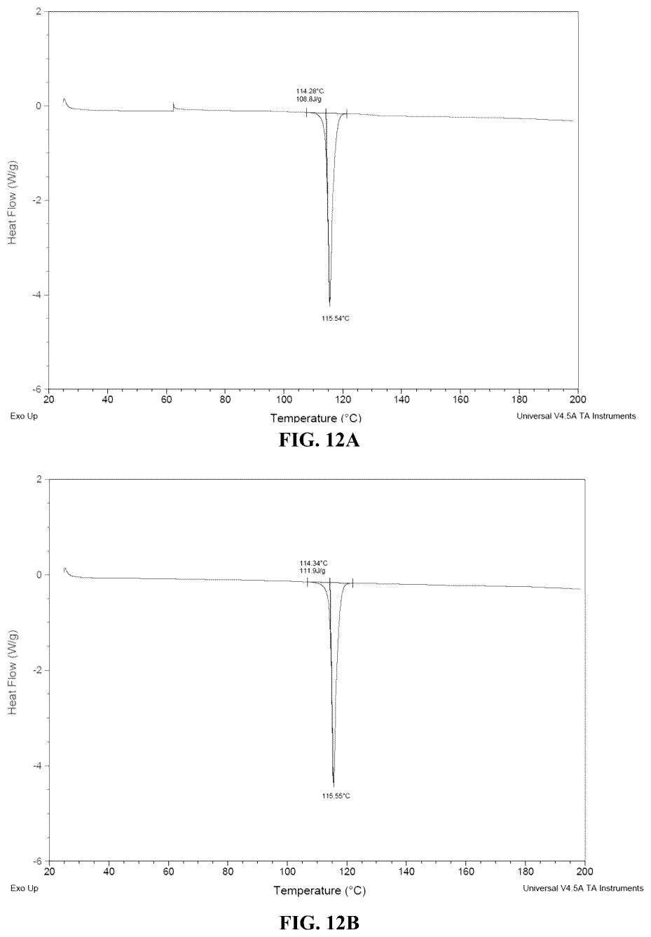

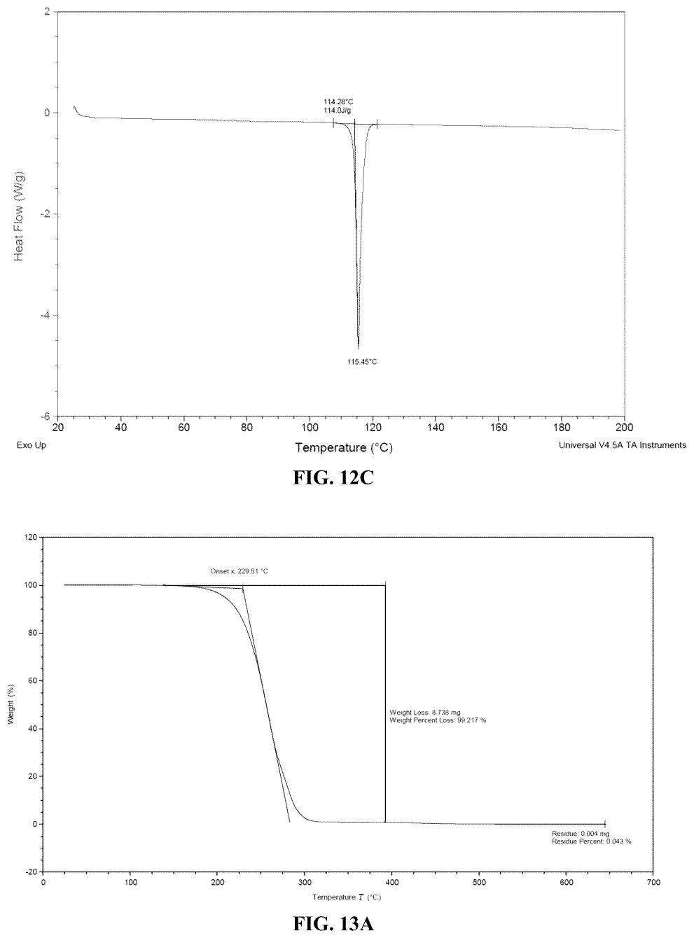

DSC curves presented herein were obtained by methods known in the art using Waters Q200. The weight of the samples was about 1 to about 5 mg. The samples were scanned up from 25° C. to 200° C. at 5° C./min increment. The present application enables the production of crystalline (+)-TBZ in various particle size ranges and crystal morphology (e.g., rod-shaped crystals and more symmetric crystals). DSC analysis of different Form 2 crystalline (+)-TBZs were demonstrated in FIGS. 12A-12C. All these DSC show an endothermic peak at 115±5° C. The sharp endothermic peak demonstrated these Form 2 crystalline (+)-TBZ were in nice and pure crystalline form.

### Example 13. Crystallization of (+)-TBZ with Tailored Size Control: Thermogravimetric Analysis (TGA)

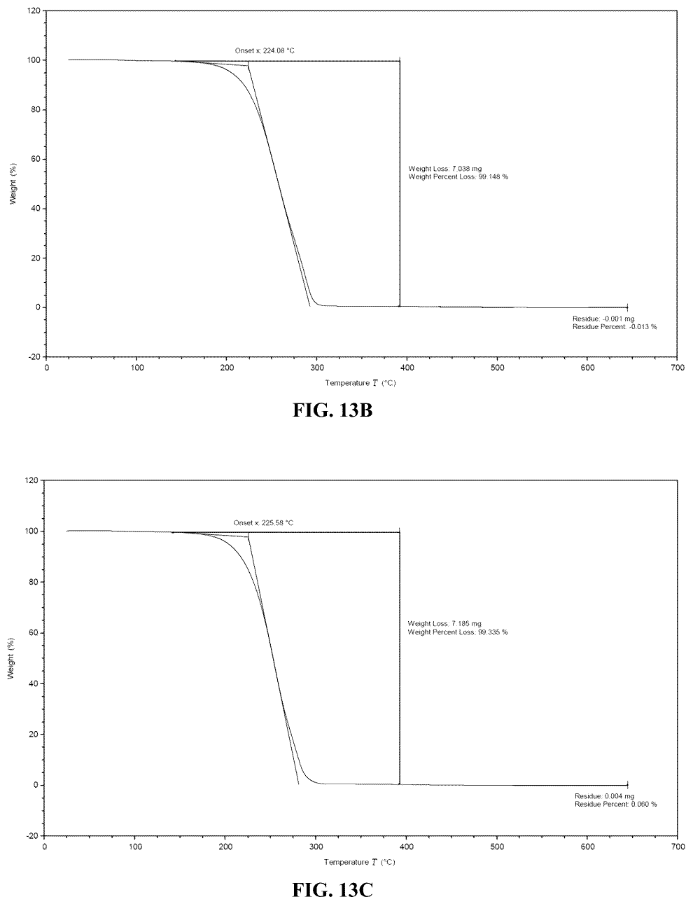

TGA curves presented herein were obtained by methods known in the art using Waters TGA 550. The weight of the samples was about 5 to about 10 mg. The samples were scanned up from 25° C. to 650° C. at 10° C./min increment. Samples were purged with nitrogen gas at a flow rate of 25 mL/min. Samples were held in standard platinum pan covered with lids. The present application enables the production of crystalline (+)-TBZ in various particle size ranges and crystal morphology (e.g., rod-shaped crystals and more symmetric crystals). TGA analysis of different Form 2 crystalline (+)-TBZs were summarized in FIGS. 13A-13C.

### Example 14. Solubility of Crystalline (+)-TBZ (Cooling Method) and TBZ Racemate

20 mg Form 2 crystalline (+)-TBZ was dispersed into 1 ml PBS with 0.2% (w/v) tween 20. Then, this excessively saturated suspension was stirred overnight at 37° C. 0.8 ml of former suspension was transferred into 1.5 mL Eppendorf centrifuge tube. The undissolved solid was spun down using bench-top centrifuge at the rotation of 14000 rpm for 5 min. The solubility was measured by the assay method described below:
Instruments: Shimadzu Separations Module (LC-30AD) with Shimadzu PDA detector (SPD-m30A).
Column: Waters Acquity UPLC BEH C18 (150×3.0 mm, 1.7 m)
Mobile Phase A: 0.5 mM, ammonium acetate: ACN=1:1
Mobile Phase B: IPA
Run time: 10 min
Elution gradient:

| Time (min) | % MPB |
|---|---|
| 0 | 35 |
| 4 | 90 |
| 6 | 95 |
| 6.5 | 35 |
| 10 | re-equilibrate |

Flow rate: 0.3 mL/min
Column temp: 55° C.
Injection volume: 2 uL
Detection: 220 nm
Run time: 10 min
(+)-TBZ retention time: ˜3.5 min

Solubility of Form 2 crystalline (+)-TBZ (cooling method) and TBZ racemate were summarized in Table 13. TBZ racemate showed about 40% less solubility, compared to Form 2 crystalline (+)-TBZ in PBS (with 0.2% w/v Tween 20).

TABLE 13
Solubility of Form 2 crystalline (+)-TBZ
(cooling method) and TBZ racemate

| Sample | Solubility (μg/mL) |
|---|---|
| TBZ racemate | 36.364 |
| crystalline (+)-TBZ | 61.032 |

### Example 15. Metabolism of Form 2 (+)-TBZ and Racemate TBZ in Rat Liver Microsome (RLM)

Rat liver microsome (RLM) study was conducted to investigate the generation of pharmaceutically active metabolites of Form 2 (+)-TBZ and TBZ racemate. As summarized in Table 14, Form 2 (+)-TBZ generated comparable amounts of therapeutically active VMAT2 inhibitors, (+)-TBZ and (+)-DHTBZ, in RLM as racemate TBZ. While commercially available treatments for hyperkinetic movement disorders use TBZ racemate and d6-TBZ racemate (such as Xenazine and Austedo), the present application demonstrates that (+)-TBZ only generates active (+)-DHTBZ and its potential to improve the treatment for hyperkinetic movement disorders to minimize side effects by avoiding the generation of inactive TBZ isoforms, such as (−)-TBZ and (−)-DHTBZ.

TABLE 14
(+)-TBZ and TBZ racemate Metabolism in RLM

| | Rat | | | | | | | | | |
|---|---|---|---|---|---|---|---|---|---|---|
| | (+)-TBZ in RLM (ng/mL) | | | | | TBZ racemate in RLM (ng/mL) | | | | |
| | Time (min) | | | | | | | | | |
| | 0 | 15 | 30 | 45 | 60 | 0 | 15 | 30 | 45 | 60 |
| (+)-TBZ | 2056 | 881 | 366 | 122 | 42 | 1892 | 957 | 477 | 199 | 83 |
| (+)-DHTBZ | 0 | 455 | 608 | 616 | 627 | 28 | 351 | 489 | 539 | 564 |
| (−)-TBZ | 0 | 0 | 0 | 0 | 0 | 2217 | 503 | 73 | 0 | 0 |
| (−)-DHTBZ | 0 | 0 | 0 | 0 | 0 | 28 | 54 | 24 | 0 | 0 |

| | | |
|---|---|---|
| Half-life (min) | 10.6 | 10.6 |

### Example 16. Sustained Release Polymeric Pharmaceutical Compositions Comprising Crystalline (+)-TBZ

Polymers can be dissolved by one or more biocompatible solvent(s) to form liquid delivery vehicles for in situ depot-forming formulations. Insoluble implants can form upon in contact with body fluid after injection, followed by continuous release of drugs controlled by diffusion and polymer degradation. Example 16 demonstrated that sustained release of (+)-TBZ can be achieved in pharmaceutical compositions comprising PLGA, Resomer RG752H (PLGA with 75:25 lactide to glycolide ratio and carboxylic acid terminal end group) and NMP at desired PLGA/NMP ratio.

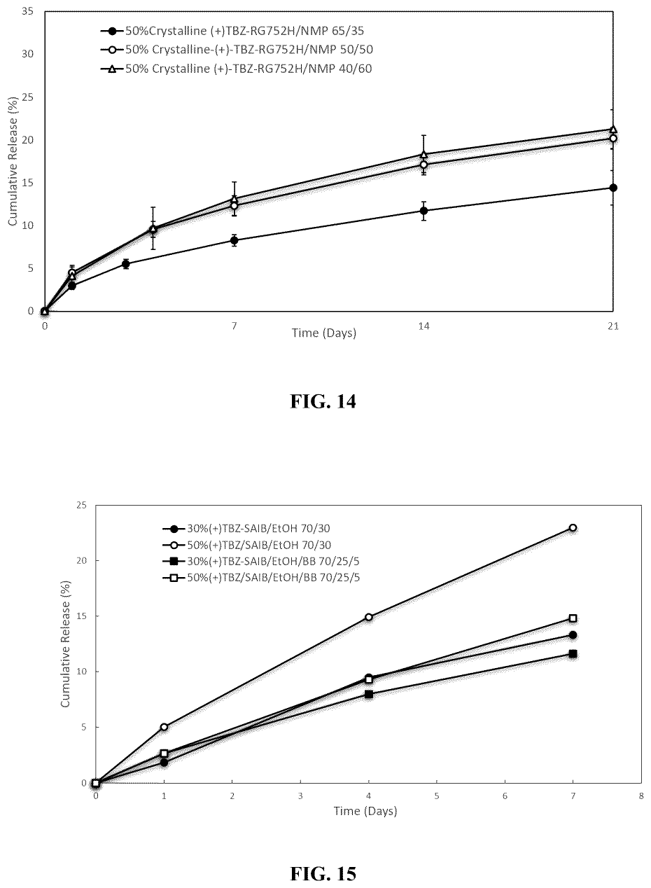

In one example, polymeric solution vehicles were first prepared by complete dissolving of Resomer RG752H in NMP at 65/35, 50/50, or 40/60 (w/w) ratio using a planetary mixer (KURABO, MAZERUSTAR). Form 2 crystalline (+)-TBZ was then blended in, mixed well to afford the final homogeneous formulations at 50% (w/w) drug loading. An aliquot of the formulation (15±5 mg) was injected into 400 mL pH 7.4 phosphate buffer saline (with 0.2% Tween 80) at 37° C. At predetermined time points, 0.5 mL of the release medium was withdrawn for HPLC analysis to calculate drug concentration in the release medium. In vitro release was performed under sink condition throughout the study. The accumulated amount of drug released was calculated at each time point. All three formulations comprising Resomer RG752H/NMP at 65/35, 50/50, and 40/60 (w/w) ratio showed sustained release for at least over 3 weeks. More or less 5% initial burst, followed by about 10% and less than 25% in vitro release were observed at 1 and 3-week (FIG. 14), respectively. Thus, the in vitro release profile of (+)-TBZ could be adjusted by selecting the beneficial compositions.

### Example 17. Sustained Release SAIB-Based Pharmaceutical Compositions Comprising of Crystalline (+)-TBZ

Sucrose acetate isobutyrate (SAIB) can be utilized as a delivery matrix for sustained delivery of drugs. Insoluble implants can form upon SAIB in contact with body fluid after injection, followed by continuous release of drugs controlled by diffusion. In one example, a pharmaceutical composition comprising crystalline (+)-TBZ, one or more biocompatible solvent(s), and SAIB was prepared for sustained release of (+)-TBZ. SAIB-based vehicle was prepared by first heating SAIB at 60° C. for about 20 minutes to reduce its viscosity, followed by mixing with one or more biocompatible solvent(s), such as ethanol (EtOH) and benzyl benzoate

(BB) at SAIB-EtOH 70/30 and SAIB/EtOH/BB 70/25/5 ratio (w/w). Form 2 crystalline (+)-TBZ was then suspended and thoroughly mixed in the above-mentioned compositions using a planetary mixer ((KURABO, MAZERUSTAR)) for 5 minutes to obtain uniform formulations at 30% or 50% (w/w) drug loading. An aliquot of the formulation (15±5 mg) was injected into 400 mL pH 7.4 phosphate buffer saline (with 0.2% Tween 80) at 37° C. At predetermined time points, 0.5 mL of the release medium was withdrawn for HPLC analysis to calculate drug concentration in the release medium. In vitro release was performed under sink condition throughout the study. The accumulated amount of drug released was calculated at each time point. FIG. 15 demonstrates the accumulated release of (+)-TBZ in different SAIB solution formulations over time. As shown in FIG. 15, less than 5% initial burst was observed for the selected crystalline (+)-TBZ-SAIB suspensions. 1-week accumulated drug release was in between 10˜25%, depending on the composition and drug loading of selected formulations. Thus, the in vitro release profile of (+)-TBZ could be adjusted by selecting the beneficial compositions.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A crystal form of (+)-tetrabenazine having an X-ray diffraction spectrum comprising peaks at diffraction 2θ angels of 8.6±0.2°, 14.1±0.2°, 15.0±0.2°, 17.3±0.2°, 22.6±0.2°, and 23.1±0.2°.

2. The crystal form of (+)-tetrabenazine according to claim 1, being Form 1 having an X-ray diffraction spectrum comprising peaks at diffraction 2θ angels of 6.5±0.2°, 8.6±0.2°, 12.1±0.2°, 14.1±0.2°, 15.0±0.2°, 16.5±0.2°, 17.3±0.2°, 17.9±0.2°, 22.6±0.2°, and 23.1±0.2°.

3. The crystal form of (+)-tetrabenazine according to claim 1, being Form 1 having an X-ray diffraction spectrum substantially as shown in FIG. 1A or FIG. 1B.

4. The crystal form of (+)-tetrabenazine according to claim 1, being Form 2 having an X-ray diffraction spectrum comprising peaks at diffraction 2θ angels of 8.6±0.2°, 12.1±0.2°, 14.1±0.2°, 15.0±0.2°, 17.3±0.2°, 17.9±0.2°, 22.6±0.2°, and 23.1±0.2°.

5. The crystal form of (+)-tetrabenazine according to claim 1, being Form 2 having an X-ray diffraction spectrum substantially as shown in FIG. 2A or FIG. 2B.

6. The crystal form of (+)-tetrabenazine according to claim 1, being Form 2 having a differential scanning calorimetry scan spectrum comprising an endothermic peak at 115±5° C.

7. The crystal form of (+)-tetrabenazine according to claim 1, being Form 2 having a thermogravimetric analysis profile substantially as shown in FIG. 13A or FIG. 13B or FIG. 13C.

8. The crystal form of (+)-tetrabenazine according to claim 1, being Form 2 having a particle size of D50 ranging from 1 μm to 200 μm, more preferably ranging from 5 μm to 150 μm, and still more preferably ranging from 10 μm to 100 μm.

9. The crystal form of (+)-tetrabenazine according to claim 1, being Form 2 having—a response angle less than 46±5 degree.

10. A process of making a crystal form of (+)-tetrabenazine having an X-ray diffraction spectrum comprising peaks at diffraction 2θ angles of 8.6±0.2°, 14.1±0.2°, 15.0±0.2°, 17.3±0.2°, 22.6±0.2°, 23.1±0.2°, comprising: allowing (±)-tetrabenazine to crystallize from a solvent selected from the group consisting of isopropyl alcohol (IPA), ethanol, acetone, ethyl acetate (EA), isopropyl acetate (IPAc), methyl tert-butyl ether (MTBE), N-methyl-2-pyrrolidone (NMP), water, a mixture or combination thereof.

11. The process according to claim 10, wherein the crystal form is Form 2 having an X-ray diffraction spectrum comprising peaks at diffraction 2θ angles of 8.6±0.2°, 12.1±0.2°, 14.1±0.2°, 15.0±0.2°, 17.3±0.2°, 17.9±0.2°, 22.6±0.2°, and 23.1±0.2°.

12. The process according to claim 10, wherein the crystal form is Form 2 having an X-ray diffraction spectrum substantially as shown in FIG. 2A or FIG. 2B.

13. The process according to claim 10, wherein (+)-tetrabenazine is allowed to crystallize at a temperature of from −15° C. to 40° C.

14. (canceled)

15. A pharmaceutical composition comprising a pharmaceutically acceptable carrier and a crystal form of (+)-tetrabenazine having an X-ray diffraction spectrum comprising peaks at diffraction 2θ angles of 8.6±0.2°, 14.1±0.2°, 15.0±0.2°, 17.3±0.2°, 22.6±0.2°, 23.1±0.2.

16. The pharmaceutical composition according to claim 14, wherein the crystal form is Form 1 having an X-ray diffraction spectrum comprising peaks at diffraction 20 angels of 6.5±0.2°, 8.6±0.2°, 12.1±0.2°, 14.1±0.2°, 15.0±0.2°, 16.5±0.2°, 17.3±0.2°, 17.9±0.2°, 22.6±0.2°, and 23.1±0.2°.

17.-19. (canceled)

20. The pharmaceutical composition according to claim 15, wherein the pharmaceutically acceptable carrier comprises a biodegradable polymer selected from the group consisting of a homopolymer polylactide or polylatic acid (PLA), a copolymer poly (lactic acid-co-glycolic acid) or poly (lactide-co-glycolide) (PLGA), and a combination thereof, preferably, wherein the PLGA has a monomer ratio of lactide: glycolide (or lactic acid: glycolic acid) between 50:50 and 99:1 inclusive.

21. The pharmaceutical composition according to claim 15, wherein the pharmaceutically acceptable carrier further comprises a pharmaceutically acceptable organic solvent selected from the group consisting of N-methyl-2-pyrrolidone, 2-pyrrolidone, N,N-dimethylformamide, dimethyl sulfoxide, benzyl alcohol, benzyl benzoate, triacetin, and combinations thereof.

22. The pharmaceutical composition according to claim 15, wherein the pharmaceutically acceptable carrier comprises a biodegradable, hydrophobic, non-polymeric carrier, preferably selected from a group of biodegradable sugar derivative, and more preferably, the biodegradable, non-polymeric, hydrophobic sugar derivative is sucrose acetate isobutyrate (SAIB).

23.-29. (canceled)
